(19) United States
(12) Patent Application Publication
(10) Pub. No.: US 2025/0121239 A1
(43) Pub. Date: Apr. 17, 2025

(54) PFAS CHEMICAL DESTRUCTION PROCESS IN ENVIRONMENTAL MEDIA

(71) Applicant: PERMA-FIX ENVIRONMENTAL SERVICES, INC., Atlanta, GA (US)

(72) Inventors: Louis F. Centofanti, Atlanta, GA (US); Tommy W. Yarbrough, Atlanta, GA (US); Jose Gabriel Garcia, Atlanta, GA (US); Christopher M. Dyer, Atlanta, GA (US)

(21) Appl. No.: 18/888,350

(22) Filed: Sep. 18, 2024

Related U.S. Application Data
(60) Provisional application No. 63/655,844, filed on Jun. 4, 2024.
(60) Provisional application No. 63/555,113, filed on Feb. 19, 2024.
(60) Provisional application No. 63/602,736, filed on Nov. 27, 2023.
(60) Provisional application No. 63/544,544, filed on Oct. 17, 2023.

(51) Int. Cl. A62D 3/34; A62D 3/40; A62D 101/22

(52) U.S. Cl. CPC A62D 3/34; A62D 3/40; A62D 2101/22; A62D 2203/02

(57) ABSTRACT

A system and method for destroying and disposing a fluorinated material, such as PFAS, with reduced emissions of gaseous PFC is provided. The method can be applied to soil or other environmental media containing the PFAS, for example at the site of the soil, either in a batch reactor or in situ. The method can include mixing PFAS, a hydroxide base, and optionally a solvent in the batch reactor to form a suspension. The reaction mixture can be heated to a temperature of 25° C. to 400° C. for about 0.5 hours to 240 hours to defluorinate the corresponding PFAS fluorocarbons and produce a defluorinated waste product. Alternatively, the suspension can be maintained for a sufficient time at room temperature. The hydroxide base and optional solvent can also be sprayed on the PFAS and optionally heated. The method converts organic fluorine present in the PFAS contaminated soil to inorganic fluoride.

### CROSS-REFERENCE TO RELATED APPLICATIONS

This U.S. non-provisional patent applications claims the benefit of and priority to U.S. provisional patent application Ser. No. 63/544,544, filed Oct. 17, 2023; U.S. provisional patent application Ser. No. 63/602,736, filed Nov. 27, 2023; U.S. provisional patent application Ser. No. 63/555,113, filed Feb. 9, 2024; and U.S. provisional patent application Ser. No. 63/655,844, filed Jun. 4, 2024, the entire disclosures of which are hereby incorporated by reference in their entirety.

### BACKGROUND OF THE INVENTION

### 1. Field of the Invention

The current invention relates generally to a system and method for destroying and disposing of fluorocarbon or fluorinated materials, for example per- and polyfluoroalkyl substances (PFAS) deposited in environmental media, for example soil, granular activated carbon (GAC) and/or biosolids.

### 2. Background Art

Fluorocarbons and fluorinated substances, such as per- and polyfluoroalkyl substances (PFAS), are anthropogenic synthetic materials used for over 90 years (1) and found in water at harmful concentrations to humans (2). PFAS have been designated as emerging contaminants of concern since 2000 (3). They can bioaccumulate in humans (4, 5, 6), have been linked to certain cancers, and have a wide range of deleterious effects including hormone and immune system interferences, ulcerative colitis, and endocrine disruption (7, 8 9), to name a few.

PFAS compose a diverse class of chemicals that, due to their low surface tension and wetting properties, are found in a wide range of products and processes, including fluoropolymers, liquid repellants for paper, packaging, textile, leather, and carpet goods, industrial surfactants, additives, coatings, and firefighting foams. (10) Fluoropolymers, for example polytetrafluoroethylene (PTFE), are believed to be the most commonly found PFAS for computer applications and surface coatings.

The United States Environmental Protection Agency (USEPA) CompTox database has identified over 9000 highly fluorinated substances with Chemical Abstracts Service numbers available in the global market, the majority being fluorinated polymers and fluorinated surfactants (11).

The most recent guideline effective on Jun. 25, 2024. has advisory limits of 4 parts per trillion (ppt) of PFOA, (12, 13) and PFOS, the USEPA added five more PFAS compounds for site cleanups (perfluoronanoic acid (PFNA), PFHxS, perfluorononanoate, perfluorooctanoate, and perfluorohexanesulfonate) based on risk-based values for regional screening levels (RSLs) (3). Various techniques have been used to decompose or dispose of hazardous substances, such as PFAS by electrochemical oxidation, direct irradiation, plasma treatment, photocatalysis, sonolysis, supercritical water oxidation, reductive hydrodefluorination and thermal degradation/incineration (14).

In 2020, the U.S. EPA (15) published a technical brief on the incineration of PFAS with the main conclusion that the effectiveness of incineration in destroying PFAS and their fate in terms of potential mixed fluorinated organic byproduct formation is not clearly understood. A significant concern is that incomplete destruction of PFAS can result in the formation of PIC (products of incomplete combustion), e.g., smaller PFAS molecules, which could be a potential hazard. Only a few studies are available related to PFAS incineration in full-scale operating facilities (16, 17-19). According to Solo-Gabriele et al., increasing incinerator temperatures decreased the total treated PFAS concentrations. However, not all PFAS species decreased with increasing temperatures (17). There is an alarming report of higher concentrations of PFOA found in the air at the incinerator sites compared to upwind sites (18). Public concern is that the incineration may spread PFAS and not break them down. This publication claims that the preliminary data show that soil and surface water near a commercial facility in Cohoes, New York, that has burned firefighting foam containing PFAS are contaminated with PFAS (20).

PFAS incineration can occur directly for PFAS-based materials, such as firefighting foams or indirectly via the incineration of waste containing PFAS, such as textiles, etc. (21). Recently the Defense Department issued a ban on incinerating PFAS-laden items, with particular emphasis on the aqueous film-forming foam often used in training and combat situations (22). In addition, under the 2022 National Defense Authorization Act (23), the military is now prohibited from incinerating PFAS-containing materials in accordance with the Clean Air Act (24). Most incineration studies monitored a limited number of compounds, leaving the question of “unmonitored” PFAS unanswered (25). Even though multiple studies were done on the thermal degradation of PFAS (26, 27, 28, 29, 30), only limited data (31, 32) is available on directly detecting degradation products during field-scale incineration. The main obstacle is still the lack of both suitable emission sampling methods (including industrial field sampling) to capture PFAS compounds and analytical methods to identify/detect PFAS and their thermal decomposition byproducts. The question remains unanswered as to how significant is the portion of volatile species that escape the analysis.

Thermal degradation/incineration is the widely available approach for managing contaminated solids, liquids, or gases using already built incinerator facilities (33), and many incineration facilities are already knowingly or unknowingly treating PFAS (e.g., consumer products, activated carbon regeneration). As previously mentioned, incineration facilities have already been deployed and are well-established in the industry.

Therefore, the initial cost of implementing the technology will be significantly reduced compared to other PFAS-destructive technologies. Together, these advantages place them as a critical solution for managing PFAS-containing waste (34, 35).

However, in general, waste byproducts in any incineration include bottom ash, which contains non-combusted products, and gas, containing tiny particles and volatile products (34). According to Wang et al. (34) regarding PFAS incineration, the resulting ash and gas are both problematic. Ash contains inorganic fluorine and remaining PFAS bound to inorganic compounds such as calcium. Ash is typically sent to a landfill or repurposed. Particulates in the gas can be captured with electrostatic precipitators. However, HF is anticipated to be the main product of PFAS thermal conversion during incineration and is a corrosive/acidic gas. Capturing or removing volatile fluoride-containing byproducts may also be problematic.

Any untreated PFAS or byproducts from incineration are released directly into the environment (34). Therefore, the potential risk of secondary air and soil pollution and the return of PFAS into the environment is very high. In addition, incomplete destruction during thermal treatment/incineration could generate an unknown array of byproducts, which might be environmentally problematic. Since current knowledge of the fate of PFAS is limited, there is concern that PFAS incineration can release toxic gases (tetrafluoromethane, hexafluoroethane, fluoro-dioxins, fluoro-benzofurans, and perfluorinated carboxylic acids) (36, 37).

Thermal treatment, such as incineration, is a popular and effective technique used to dispose of hazardous substances in land-scarce and resource-lacking locations because it can greatly reduce volume of the waste and can produce electricity. However, a significant concern associated with incineration of the waste is emission of harmful gases. Dioxin and furan are commonly studied gases because they are serious health hazards. There are few studies on the production of perfluorinated compound (PFC) gases from the thermal treatment process. However, it was reported that thermal decomposition of PFAS can form gas-phase PFCs, such as CF₄ and C₂F₆, which are harmful to the environment. Gaseous PFCs are potent greenhouse gases. The global warming potential of CF₄ is 6500 times that of CO₂, and the atmospheric lifetime of C₂F₆ is 50,000 years. Due to the long atmospheric lifetime of gaseous PFCs, gaseous PFC emissions can permanently alter the radiative budget of the atmosphere. Other methods for disposing PFAS are being investigated, but fluorinated halogenated hydrocarbons, such as those found in PFAS, are very difficult to destroy due to the strength of the carbon-fluorine bond. There remains a need for a more sustainable method for disposing of PFAS, especially PFAS contained in “Environmental Media”.

### SUMMARY

One aspect of the disclosure provides a method for destroying and disposing a fluorinated material, for example a polyfluoroalkyl substance (PFAS). The method includes placing environmental media, containing the fluorinated material in a batch reactor along with a hydroxide base and optionally a solvent system comprised of diglyme, polyethers, polyether alcohols, any of the polyethylene glycols selected from ethylene glycol, PEG50 through PEG3350, N-methylpyrrolidine, cyrene or water (“solvent”) to form a suspension, and maintaining the fluorinated material in the batch reactor until a defluorinated waste product is produced.

According to an example embodiment, the environmental media containing the fluorinated material is soil, granular activated carbon (GAC), biosolids, or other media found in the environment and is referred to as “Environmental Media”. According to this example, the batch reactor is located at the site of the “Environmental Media”. The batch reactor can be located in a mobile unit.

The fluorinated material can be maintained in the batch reactor at room temperature or the fluorinated material can be heated in the batch reactor.

According to an example embody, the fluorinated material, the hydroxide base, and optionally the “solvent” are allowed to react with one another in the batch reactor for a time of about 0.5 hours to about 240 hours; and the heating step includes heating the suspension to a temperature ranging from about 25° C. to about 400° C.

According to another example embodiment, the hydroxide base is potassium hydroxide; the heating step includes heating to a temperature of about 180° C.; the fluorinated material is PFAS; the PFAS, the hydroxide base, and the “solvent” are placed in the batch reactor and allowed to react with one another for a time of about 4 hours to about 8 hours.

The hydroxide base can include at least one of potassium hydroxide (KOH), calcium hydroxide (Ca(OH)₂), cesium hydroxide (CsOH), lithium hydroxide (LiOH), sodium hydroxide (NaOH), strontium hydroxide (Sr(OH)₂) and mixtures thereof. The “solvent” can be added to the batch reactor and can include at least one of diglyme, polyethylene glycol ether, cyrene, N-methylpyrrolidone, and deionized water.

Another aspect of the disclosure provides a method for destroying and disposing a fluorinated material which includes spraying a hydroxide base and optionally a “solvent” on a fluorinated material, such as PFAS.

The fluorinated material is contained in environmental media. For example, the spraying step can be applied to the “Environmental Media” containing the fluorinated material, and the spraying step is performed at the site of the “Environmental Media”. According to an embodiment, the method can include heating the fluorinated material after the spraying step.

Another aspect of the disclosure provides a batch reactor for disposing a fluorinated material contained in environmental media according to the method disclosed herein. And the fluorinated material is PFAS.

### BRIEF DESCRIPTION OF THE DRAWING

Other advantages of the present invention will be readily appreciated, as the same becomes better understood by reference to the following detailed description when considered in connection with the accompanying drawing wherein:

FIG. 1 illustrates a batch system for destroying and defluorinating a fluorocarbon present in PFAS, specifically an “Environmental Media” contaminated with PFAS to produce a defluorinated waste product according to an example embodiment.

### DETAILED DESCRIPTION OF EXAMPLE EMBODIMENTS

The materials, compounds, compositions, and methods described herein may be understood more readily by reference to the following detailed description of specific aspects of the disclosed subject matter and the examples included therein.

Before the present materials, compounds, compositions, and methods are disclosed and described. It is to be understood that the aspects described below are not limited to specific methods of specific reagents, as such may, of course, vary. It is also to be understood that the terminology used herein is for the purpose of describing only and is not intended to be limiting.

Also, throughout this specification, various publications are referenced. The disclosures of these publications are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and the claims that follow, reference will be made to several terms, which shall be defined to have the following meanings:

Throughout the specification and claims the word “comprise” and other forms of the word, such as “comprising” and “comprises,” means including but not limited to, and is not intended to exclude, for example, other additives, “solvent”, bases, components, integers, or steps; As used herein, the singular forms “a,” “an,” and “the” include singular or plural referents unless the context clearly dictates otherwise. Thus, for example, reference to “a composition” includes mixtures of two or more such compositions. “Optional” or “optionally” means that the subsequently described event or circumstance can or cannot occur, and that the description includes instances where the event or circumstance occurs and instances where it does not. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.; As used herein, the term “composition” is intended to encompass a product comprising the specified ingredients in the specified amounts, as well as any product which results, directly or indirectly, from combination of the specified ingredients in the specified amounts. References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5 and are present in such ratio regardless of whether additional components are contained in the mixture. A weight percent (wt. %) of a component, unless specifically stated to the contrary, is based on the total weight of the formulation or composition in which the component is included. As used herein, the term “substituted” is contemplated to include all permissible substituents of inorganic base compounds. In a broad aspect, the permissible substituents include all alkali and alkaline-earth metals in the periodic table. Illustrative substituents include, for example, those described below. The permissible substituents can be one or more and the same or different for appropriate inorganic base compounds.

Those persons of ordinary skill in the art will appreciate that Compounds of Formula I are examples of inorganic base analogs. As used herein, “an analog of potassium hydroxide” or “analogs of potassium hydroxide” are not limited to those analog compounds represented by Formula I, and may include many additions or substitutions of elements, groups, or moieties to the chemical structure of potassium hydroxide.

M(OH)x Formula I

wherein x is the number of hydroxy units per M valence; and
M is selected from the alkali or alkaline-earth metal groups.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

One aspect of the invention provides a system and method for disposing of per- and polyfluoroalkyl substances (PFAS) with reduced emissions of gaseous PFC, such as CF₄ and C₂F₆. In certain embodiments, the PFAS is a single per- and polyfluorinated compound or a mixture of several per- and polyfluorinated compounds. The current invention also pertains to a method of adding a “solvent” to the PFAS and applying several heating temperatures in the degradation process. More specifically, the subject matter disclosed herein relates to a system and method that can be used for reducing emissions of gaseous perfluorinated compounds (PFCs) during thermal treatment of PFAS.

Various types of PFAS can be treated with the batch system according to the present invention, for example perfluorooctanoic acid (PFOA). Although the system and method are typically applied to PFAS, and PFAS will be discussed throughout the present disclosure, the system and method can be used to dispose of any type of fluorocarbon or fluorinated material. The system and method destroy the carbon-fluorine bonds and converts the organic fluorine present in the fluorocarbon or other fluorinated material to inorganic fluoride.

According to example embodiments, disclosed herein the PFAS or other fluorinated material is found in environmental media.

The method for destroying and disposing the fluorinated material, or the environmental media containing the fluorinated material, such as PFAS, first includes placing the PFAS contaminated “Environmental Media” in a batch system, more specifically in a batch reactor. A hydroxide base is then added, for example potassium hydroxide (KOH), calcium hydroxide (Ca(OH)₂), cesium hydroxide (CsOH), lithium hydroxide (LiOH), strontium hydroxide (Sr(OH)₂) and/or sodium hydroxide (NaOH). A “solvent” is also optionally added to the batch reactor to form a sludge/suspension. Water may also be present optionally as a co-“solvent” in the batch reactor.

The PFAS contaminated medium is typically maintained in the batch reactor at a temperature of ranging from room temperature for several days, or 100° C. and 200° C. for at least 2 hours, for example 3 to 5 hours, or up to 8 hours to defluorinate the PFAS contaminated “Environmental Media” and produce a defluorinated waste product consisting of inorganic fluoride. Some types of PFAS, such as perfluorooctyl sulfonate (PFOS), may require higher temperatures and longer times in the reactor, for example temperatures up to but not limited to 300° C. According to other embodiments, the temperature of the batch system may be less than 100° C., for example room temperature or 50° C. up to 100° C. When the temperature of the batch system is lower, the time required to defluorinate the PFAS in contaminated “Environmental Media” and produce a defluorinated waste product consisting of an inorganic fluoride is longer. The defluorinated waste product produced may typically include polyethylene glycol and/or the “solvent” used in the reactor, formate, carbonate, oxalate and/or glycolate organic salts, and inorganic fluoride(s) wherein the composition of the inorganic fluoride, i.e. potassium fluoride, sodium fluoride, lithium fluoride, strontium fluoride and/or calcium fluoride or combinations thereof, etc., depends on the hydroxide base or mixture of hydroxide bases used in the batch system, The defluorinated waste product can be further incinerated without significant emissions of the harmful gaseous PFCs.

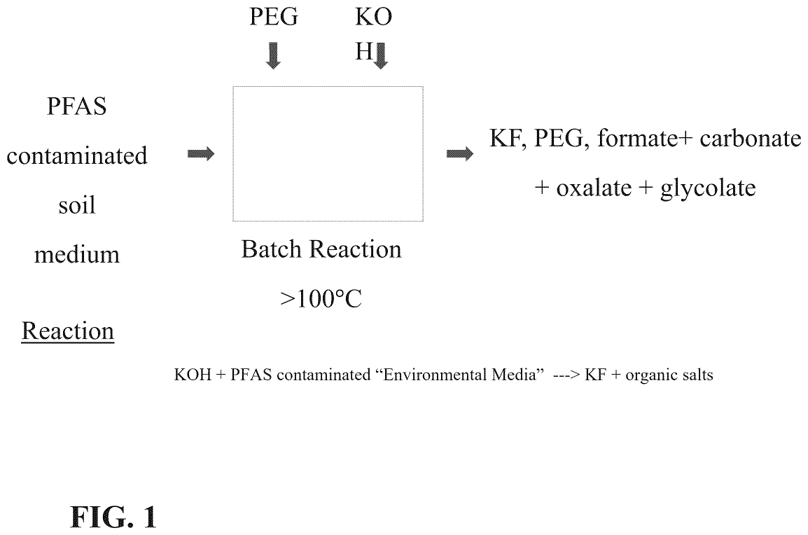

FIG. 1 illustrates the batch system and method according to an example embodiment. According to this example, “Environmental Media” containing PFAS is placed in the batch reactor along with PEG and potassium hydroxide (KOH). The PEG is PEG200 which has a molar mass of 190-210 g/mol and a chemical formula of H—(O—CH₂CH₂)ₙ—OH, where n=8.2 to 9.1. It is believed that the PEG200 could be replaced optionally with diglyme, polyethers, polyether alcohols, N-methylpyrrolidine, cyrene, water and/or polyethylene glycol selected from ethylene glycol and/or PEG50 through PEG3350. The KOH could be replaced with another hydroxide base comprised of but not limited to sodium, calcium, lithium, strontium or cesium or optionally mixtures thereof, etc. According to this example, the PFAS contaminated “Environmental Media” is allowed to react in the batch system at a temperature of 180° C. to 200° C. for approximately 4 hours at ambient pressure. The resulting defluorinated waste product includes the product generated potassium fluoride (KF), PEG200, unreacted excess potassium hydroxide (KOH), and carbonate and/or formate and/or oxalate and/or glycolate organic salts or mixtures thereof. The chemical reaction taking place in the batch example system of FIG. 1 includes:

nKOH+PFAS contaminated“Environmental Media”→qKF+organic salts

After the batch process, the defluorinated waste product can be thermally treated, for example by incineration, with reduced emissions of the hazardous gaseous PFCs, such as CF₄ and C₂F₆.

Before incineration, some of the components present in the defluorinated waste product can be recycled or removed and disposed of without thermal treatment. For example, according to one embodiment, the PEG200 is removed from the defluorinated waste product and recycled. The recycled PEG200 can be used in future batch systems.

Another aspect of the invention is the capability of reusing the unreacted components in the process of defluorination of the PFAS contaminated “Environmental Media”.

As indicated above, the system and method for treating, destroying and disposing per- and polyfluoroalkyl substances (PFAS) with reduced emissions of gaseous PFCs can be applied to “Environmental Media” containing PFAS.

According to one embodiment, the treatment of the PFAS contaminated “Environmental Media” is conducted at the location of the PFAS contaminated “Environmental Media” (“on site”) using the system and methods described herein. For example, the treatment can be conducted at the site of a generator. A mobile unit containing the batch reactor is located at the site of the “Environmental Media” or taken to the site of the “Environmental Media”. As described above, the “Environmental Media” containing the PFAS is added to the batch reactor along with a hydroxide base (strong base) and optionally a “solvent”. The “Environmental Media” containing the PFAS, hydroxide base, and optional “solvent” is maintained in the batch reactor at a temperature ranging from room temperature for several days, or 100° C. and 200° C. for at least 2 hours, for example 3 to 5 hours, or up to 8 hours, to defluorinate the PFAS and produce a defluorinated waste product consisting of inorganic fluoride.

According to another embodiment, the treatment is conducted on site by spraying the hydroxide base and optionally the “solvent” directly on the “Environmental Media” containing the PFAS. For example, the spray can be applied to contaminated “Environmental Media” which is still located on the ground. After the spraying step, the contaminated “Environmental Media” can be covered, if necessary, and the hydroxide base and optional “solvent” remains on the contaminated “Environmental Media” for several weeks. The contaminated “Environmental Media” can also be heated after the treatment step if desired. After several weeks, the “Environmental Media” is tested to determine if a proper (reduced) level of PFAS has been achieved. If the required level has not been achieved, then more of the hydroxide base and optional “solvent” can be added until the proper level is achieved.

The on-site treatment of the contaminated “Environmental Media” described above provides several advantages, primary reduced time and costs, since the contaminated “Environmental Media” does not need to be transported to an off site facility.

### Experiment

### General Procedure for the Defluorination Reaction.

PFAS contaminated “Environmental Media” was treated with a hydroxide base (sodium hydroxide, potassium hydroxide or calcium hydroxide or combinations thereof in various ratios) either neat or in the presence of a “solvent” in various ratios at 150° C. to 200° C. for 4 hours. The resulting reaction mixture is allowed to cool to room temperature. The reaction material is analyzed by 19F NMR.

Representative example 1. “solvent” assisted.

In a 40 mL vial with a screwcap, to a Gainesville land soil (14.59 g/1 part), a solution of AFFF (at 750 ppm PFAS) in DIW:PEG200 (1:1) is added followed by crushed potassium hydroxide pellets (1.44 g/10% w/w). The vial is immersed in an oven at 70° C. and allowed to react for 8 hours. The resulting suspension in an amber colored reaction liquid is allowed to cool to room temperature, sonicated for 15 minutes followed by centrifugation at 3000 rpm for 15 minutes and decanted. The decantate is analyzed by ¹⁹F NMR. This reaction solution shows the presence of only inorganic potassium fluoride.

In summary, in accordance with the purposes of the disclosed materials and methods, as embodied and broadly described herein, the disclosed subject matter, in one aspect, relates to the composition and methods of defluorination or destroying of PFAS contaminated “Environmental Media” generating inorganic fluoride in the form of a salt. Moreover, it relates to methods of reducing emissions of gaseous perfluorinated compounds (PFCs) during thermal treatment of PFAS contaminated “Environmental Media”. In specific aspects, the disclosed subject matter relates to the selection of materials for a greener process.

Certain embodiments of this invention provide a composition comprising at least one “solvent” and a strong base; and wherein the hydroxide base is comprised of potassium hydroxide, sodium hydroxide, caesium hydroxide, lithium hydroxide, strontium hydroxide and/or calcium hydroxide either by themselves or in combination at different compositions in w/w % according to reaction scheme I.

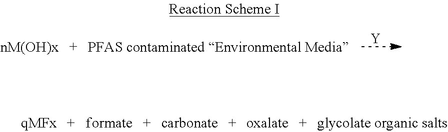

wherein;
n is the minimum amount of molar equivalents to degrade the organic fluorine in PFAS;
M is comprised of potassium, sodium, cesium, lithium, strontium or calcium;
q is the maximum amount of molar equivalents generated by degradation of the organic fluorine in the PFAS contaminated “Environmental Media”;
x is the number of hydroxy and fluoride units per M valence; and,
Y is comprised of “solvent”.

In another embodiment, these compositions as described hereinabove, do not include addition of said solvent according to the reaction scheme II.

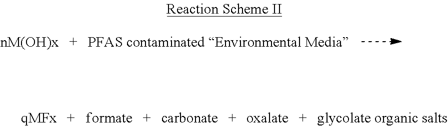

wherein;
n is the minimum amount of molar equivalents to degrade the organic fluorines in PFAS contaminated “Environmental Media”;
M is comprised of potassium, sodium, cesium, lithium, strontium or calcium;
q is the maximum amount of molar equivalents generated by degradation of the organic fluorine in PFAS contaminated “Environmental Media”; and,
x is the number of hydroxy and fluoride units per M valence;

Obviously, many modifications and variations of the present invention are possible in light of the above teachings and may be practiced otherwise than as specifically described while within the scope of the following disclosure and claims.

### LITERATURE CITED

1. Meegola, J. N. et al. Int. J. Environ. Res. Public Health 2022, 19(24), 16397.
2. Ebnesajjad, S. Introduction to Fluoropolymers; Elsevier: Amsterdam, The Netherlands, 2013, 17-35.
3. Camdzic, D. et al. J. Hazard. Mater. Lett. 2021, 2, 100023. https://www.sciencedirect.com/science/article/pii/S2666911021000113 #bib0285
4. Arana Juve, J.-M. et al. Curr. Opin. Chem. Engin. 2023, 41, 100943.
5. Buck, R. C. et al. Integr. Environ. Assess. Manag., 2011, 7(4), 513-541.
6. Ying, L. Occup. Environ. Med., 2018, 75(1), 46-51.
7. Kelly, B. C. et al. Environ. Sci. Technol., 2009, 43(11), 4037-4043.
8. Jiang, Q. et al. Toxicological Effects of Perfluoroalkyl and Polyfluoroalkyl Substances 2015, 177-201.
9. Abbott, B. D. et al. Reprod. Toxicol., 2012, 33(4), 491-505.
10. Leeson, A. et al., Environ. Toxicol. Chem. 2021. 40, 24-36.
11. Wang, Z. et al., Environ. Int. 2013, 60, 242-8.
Vierke, L. et al., Environ. Sci. Eur. 2012, 24, 16.
Leung, S. C. E. et al., Sci. Total Environ. 2022, 827, 153669. https://comptox.epa.gov/dashboard/chemical-lists/PFASSTRUCT;

https://comptox.epa.gov/dashboard/chemical-lists/PFASDEV
12. Krafft, M. P. et al. Curr. Opin. Colloid Interface Sci. 2015, 20, 192-212.
Ojo, A. F. et al. J. Hazard. Mater. 2021, 407, 12486.
13. https://www.whitehouse.gov/wp-content/uploads/2023/03/OSTP-March-2023-PFAS-Report.pdf https://www.epa.gov/sdwa/and-polyfluoroalkyl-substances-pfas
14. Int. J. Environ. Res. Public Health 2022, 19(24), 16397
15. EPA. Per-and Polyfluoroalkyl Substances (PFAS): Incineration to Manage PFAS Waste Streams Background. US EPA Tech. Brief. 2020.
16. Water Res. 2007, 41, 4611-4620
17. Waste Manag. 2020, 107, 191-200
18. Sci. Total Environ. 2020, 705, 135832.
19. Water Environ. Res. 2021, 93, 826-843
20. https://cen.acs.org/environment/persistent-pollutants/Incincerators-spread-break-down-PFAS/98/web/2020/04
21. Stoiber, T.; Evans, S.; Naidenko, O. V. Disposal of products and materials containing per- and polyfluoroalkyl substances (PFAS): A cyclical problem. Chemosphere 2020, 260, 127659.
22. Crunden, E. A. Defense Department Hits the Brakes on PFAS Incineration. Available online: https://www.eenews.net/articles/defense-department-hits-the-brakes-on-pfas-incineration/23.
23. U.S.C. H. R.2591 PFAS Waste Incineration Ban Act of 2019. 2019. Available online: https://www.govinfo.gov/24.
24. U.S.C. Public Law 116-92-National Defense Authorization Act for Fiscal Year 2020. 2019. Available online: https://www.govinfo.gov/
25. Horst, J.; McDonough, J.; Ross, I.; Houtz, E. Understanding and managing the potential by-products of PFAS destruction. Groundw. Monit. Remediat. 2020, 40, 17-27.
26. Stockenhuber, S.; Weber, N.; Dixon, L.; Lucas, J.; Grimison, C.; Bennett, M.; Stockenhuber, M.; Mackie, J.; Kennedy, E. Thermal Degradation of Perfluorooctanoic Acid (PFOA). In Proceedings of the 16th International Conference on Environmental Science and Technology, Rhodes, Greece, 4-7 Sep. 2019.
27. Altarawneh, M.; Almatarneh, M. H.; Dlugogorski, B. Z. Thermal decomposition of perfluorinated carboxylic acids: Kinetic model and theoretical requirements for PFAS incineration. Chemosphere 2022, 286, 131685.
28. Sasi, P. C.; Alinezhad, A.; Yao, B.; Kubátová, A.; Golovko, S. A.; Golovko, M. Y.; Xiao, F. Effect of granular activated carbon and other porous materials on thermal decomposition of per- and polyfluoroalkyl substances: Mechanisms and implications for water purification. Water Res. 2021, 200, 117271.
29. Xiao, F.; Sasi, P. C.; Yao, B.; Kubátová, A.; Golovko, S. A.; Golovko, M. Y.; Soli, D. Thermal decomposition of PFAS: Response to comment on “thermal stability and decomposition of perfluoroalkyl substances on spent granular activated carbon”. Environ. Sci. Technol. Lett. 2021, 8, 364-365.
30. Crownover, E.; Oberle, D.; Kluger, M.; Heron, G. Perfluoroalkyl and polyfluoroalkyl substances thermal desorption evaluation. Remediat. J. 2019, 29, 77-81.
31. Yamada, T.; Taylor, P. H.; Buck, R. C.; Kaiser, M. A.; Giraud, R. J. Thermal degradation of fluorotelomer treated articles and related materials. Chemosphere 2005, 61, 974-984.
32. Taylor, P. H.; Yamada, T.; Striebich, R. C.; Graham, J. L.; Giraud, R. J. Investigation of waste incineration of fluorotelomer-based polymers as a potential source of PFOA in the environment. Chemosphere 2014, 110, 17-22.
33. Vidonish, J. E.; Zygourakis, K.; Masiello, C. A.; Sabadell, G.; Alvarez, P. J. J. Thermal treatment of hydrocarbon-impacted soils: A review of technology innovation for sustainable remediation. Engineering 2016, 2, 426-437.
34. Wang, J.; Lin, Z.; He, X.; Song, M.; Westerhoff, P.; Doudrick, K.; Hanigan, D. Critical review of thermal decomposition of per- and polyfluoroalkyl substances: Mechanisms and implications for thermal treatment processes. Environ. Sci. Technol. 2022, 56, 5355-5370.
35. Aleksandrov, K.; Gehrmann, H. J.; Hauser, M.; Mätzing, H.; Pigeon, D.; Stapf, D.; Wexler, M. Waste incineration of polytetrafluoroethylene (PTFE) to evaluate potential formation of per- and poly-fluorinated alkyl substances (PFAS) in Flue Gas. Chemosphere 2019, 226, 898-906. [
36. García, A. N.; Viciano, N.; Font, R. Products obtained in the fuel-rich combustion of PTFE at high temperature. J. Anal. Appl. Pyrolysis 2007, 80, 85-91.
37. Wang, F.; Shih, K.; Lu, X.; Liu, C. Mineralization behavior of fluorine in perfluorooctanesulfonate (PFOS) during thermal treatment of lime-conditioned sludge. Environ. Sci. Technol. 2013, 47, 2621-2627.

It will be appreciated by those persons skilled in the art that changes could be made to embodiments of the present invention described herein without departing from the broad inventive concept thereof. It is understood, therefore, that this invention is not limited by any particular embodiments disclosed but is intended to cover the modifications that are within the spirit and scope of the invention, as defined by the appended claims.

What is claimed is:

1. A method for destroying and disposing a fluorinated material, comprising the steps of:
placing environmental media containing a fluorinated material in a batch reactor along with a hydroxide base and optionally a solvent, comprised of diglyme, polyethers, polyether alcohols, N-methylpyrrolidine, cyrene, water, and/or polyethylene glycol selected from ethylene glycol and/or one or more of PEG50 through PEG3350, to form a suspension, and maintaining the fluorinated material in the batch reactor until a defluorinated waste product is produced.

2. The method of claim 1, wherein the fluorinated material is any polyfluoroalkyl substance (PFAS).

3. The method of claim 1, wherein the environmental media containing the fluorinated material is soil.

4. The method of claim 3, wherein the batch reactor is located at the site of the soil.

5. The method of claim 1, wherein the environmental media containing the fluorinated material is GAC.

6. The method of claim 5, wherein the batch reactor is located at the site of the GAC.

7. The method of claim 1, wherein the environmental media containing the fluorinated material is biosolids.

8. The method of claim 7, wherein the batch reactor is located at the site of the biosolids.

9. The method of claim 4, wherein the batch reactor is located in a mobile unit.

10. The method of claim 6, wherein the batch reactor is located in a mobile unit.

11. The method of claim 8, wherein the batch reactor is located in a mobile unit.

12. The method of claim 1, wherein the fluorinated material is maintained in the batch reactor at room temperature.

13. The method of claim 1, wherein the fluorinated material is heated in the batch reactor.

14. The method of claim 13, wherein the fluorinated material, the hydroxide base, and optionally the solvent are allowed to react with one another in the batch reactor for a time of about 0.5 hours to about 240 hours; and the heating step includes heating the suspension to a temperature ranging from about 25° C. to about 400° C.

15. The method of claim 13, wherein the hydroxide base is potassium hydroxide; the heating step includes heating to a temperature of about 180° C.; the fluorinated material is PFAS; the PFAS, the hydroxide base, and the solvent are placed in the batch reactor and allowed to react with one another for a time of about 4 hours to about 8 hours.

16. The method of claim 1, wherein the hydroxide base includes at least one of potassium hydroxide (KOH), calcium hydroxide (Ca(OH)₂), cesium hydroxide (CsOH), lithium hydroxide (LiOH), sodium hydroxide (NaOH), strontium hydroxide (Sr(OH)₂) and mixtures thereof.

17. The method of claim 1, wherein the solvent comprised of polyethers, polyether alcohols, any of the polyethylene glycols selected from diglyme, polyethers, polyether alcohols, N-methylpyrrolidine, cyrene, water, and/or polyethylene glycol selected from ethylene glycol and/or one or more of PEG50 through PEG3350, is added to the batch reactor and includes at least one of polyethylene glycol selected from ethylene glycol, diglyme, and/or PEG50 through PEG3350.

18. The method of claim 17, wherein the preferred solvent is PEG200.

19. A method for destroying and disposing a fluorinated material, comprising spraying a hydroxide base and optionally a solvent on a fluorinated material.

20. The method of claim 19, wherein the fluorinated material is PFAS.

21. The method of claim 19, wherein the spraying step is applied to environmental media containing the fluorinated material.

22. The method of claim 21, wherein the environmental media is soil.

23. The method of claim 16, wherein the spraying step is performed at the site of the soil.

24. The method of claim 21, wherein the environmental media is GAC.

25. The method of claim 16, wherein the environmental media is GAC.

26. The method of claim 21, wherein the environmental media is biosolids.

27. The method of claim 16, wherein the environmental media is biosolids.

28. The method of claim 19 including heating the fluorinated material after the spraying step.

29. A system comprising a batch reactor for disposing a fluorinated material according to the method of claim 1.

30. The system of claim 29, wherein the environmental media is soil.

31. The system of claim 29, wherein the environmental media is GAC.

32. The system of claim 9, wherein the environmental media is biosolids.

33. The system of claim 29, wherein the fluorinated material is any per- and polyfluoroalkyl substance (PFAS).
